(19) United States
(12) Patent Application Publication
(10) Pub. No.: US 2009/0311137 A1
(43) Pub. Date: Dec. 17, 2009

(54) ATOMIZER USING ELECTROLYZED LIQUID AND METHOD THEREFOR

(75) Inventor: Bruce F. Field, Golden Valley, MN (US)

Correspondence Address: WESTMAN CHAMPLIN & KELLY, P.A., SUITE 1400, 900 SECOND AVENUE SOUTH, MINNEAPOLIS, MN, 55402, US

(73) Assignee: Tennant Company, Minneapolis, MN (US)

(21) Appl. No.: 12/481,098

(22) Filed: Jun. 9, 2009

Related U.S. Application Data
(60) Provisional application No. 61/060,684, filed on Jun. 11, 2008.

(51) Int. Cl. A61L 2/18; B05B 17/04

(52) U.S. Cl. 422/28; 239/102.2

(57) ABSTRACT

An apparatus and method are provided for generating an electrochemically activated liquid that is rendered at least partially aerosol.

### CROSS-REFERENCE TO RELATED APPLICATION(S)

The present application claims priority to U.S. Provisional Application No. 61/060,684, filed on Jun. 11, 2008, and entitled “ATOMIZER USING ELECTROLYZED LIQUID AND METHOD THEREFOR”, the disclosure of which is incorporated by reference in its entirety.

### FIELD OF THE DISCLOSURE

The present disclosure relates to sanitizing with electrochemically activated liquids and, more particularly, to an apparatus and method of sanitizing.

### BACKGROUND

Atomization is used in a variety of commercial and industrial applications, such as humidification applications, medical applications (e.g., inhalers), and coating applications. Atomization involves converting a bulk liquid into a spray or mist by passing the liquid through a nozzle, and rendering the liquid aerosol. The aerosol droplets of the liquid are suspended in the environmental gas (e.g. air). Despite the wide variety of applications for atomizers, there is an ongoing need for increased atomization applications.

### SUMMARY

An aspect of the disclosure is directed to an atomizer assembly that includes an electrolysis cell configured to electrochemically activate a liquid, and an atomizer configured to render the electrochemically-activated liquid at least partially aerosol.

Another aspect of the disclosure is directed to a method for decontaminating an environment. The method includes electrochemically activating a liquid, and rendering the electrochemically-activated liquid at least partially aerosol.

A further aspect of the disclosure is directed to a method for decontaminating an environment, where the method includes introducing a first portion of a feed liquid into an anode chamber of an electrolysis cell, introducing a second portion of the feed liquid into a cathode chamber of the electrolysis cell, and applying a voltage potential across the first and second portions of the feed water to electrochemically activate the first and second portions of the feed liquid. The method further includes feeding at least one of the first and second electrochemically-activated portions of the feed liquid to an atomizer, and rendering the at least one electrochemically-activated portion at least partially aerosol

### BRIEF DESCRIPTION OF THE DRAWINGS

FIG. 1 is a simplified, schematic diagram of an atomizer assembly according to an exemplary aspect of the present disclosure.

FIG. 2 illustrates an example of an electrolysis cell having an ion-selective membrane.

FIG. 3 illustrates an electrolysis cell having no ion-selective membrane according to a further example of the disclosure.

### DETAILED DESCRIPTION

An aspect of the present disclosure relates to systems and methods for sanitizing and decontaminating environments and/or objects with the use of electrochemically-activated liquid (e.g., water) in the form of an alkaline liquid, an acidic liquid, or a blended combination of the alkaline and acidic species. The electrochemically-activated (EA) liquid may be atomized to eliminate or reduce the concentration of contaminants in the air and on the surfaces of an environment (e.g., a room or other generally enclosed area).

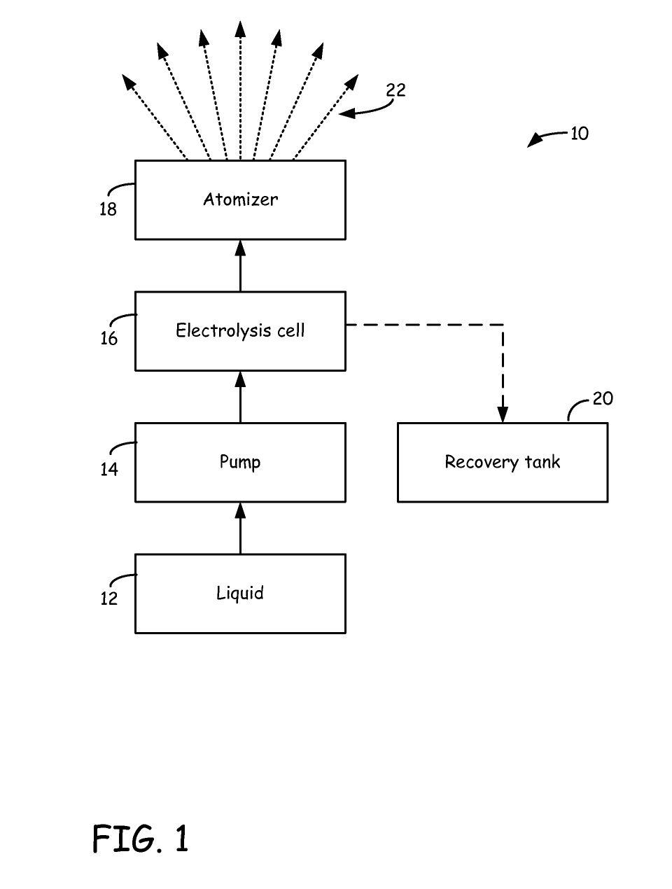

In one aspect of the disclosure, an electrolysis cell is used to produce an electrochemically-activated liquid, which is then atomized to produce a working spray. FIG. 1 is a simplified, schematic diagram of atomizer assembly 10 according to an exemplary aspect of the present disclosure. Atomizer assembly 10 includes reservoir 12, which may include one or more vessels for retaining a supply of liquid for use with atomizer assembly 10. In additional and alternative embodiments, atomizer assembly 10 may include a fitting or other liquid input for containing and/or receiving a working liquid to be treated and then atomized. In an example, the liquid to be treated includes an aqueous composition, such as regular tap water.

Atomizer assembly 10 further includes pump 14, one or more electrolysis cells 16, atomizer 18, and recovery tank 20. Although not shown in FIG. 1, atomizer assembly 10 can also include other elements, such as a power source (e.g., a battery or power cord), one or more control switches, and control electronics for controlling operation of the pump 14, electrolysis cell 16 and atomizer 18.

Pump 14 includes one or more liquid pumps configured to relay the liquid from reservoir 12 to electrolysis cell 16. Pump 14 can be located upstream or downstream of electrolysis cell 16 (shown as being upstream in FIG. 1). When energized, pump 14 draws liquid from reservoir 12, through electrolysis cell 18, and into atomizer 18.

Electrolysis cell 16 is a fluid treatment cell that is adapted to apply an electric field across water between at least one anode electrode and at least one cathode electrode. Suitable cells for electrolysis cell 16 may have any suitable number of electrodes, and any suitable number of chambers for containing the water. As discussed below, electrolysis cell 16 may include one or more ion exchange membranes between the anode and cathode, or can be configured without ion exchange membranes. Electrolysis cell 16 may have a variety of different structures, such as, but not limited to those disclosed in Field et al., U.S. Patent Publication No. 2007/0186368, published Aug. 16, 2007. In an alternative embodiment, atomizer assembly 10 may include multiple electrolysis cells 16 that operate in series and/or parallel arrangements to electrochemically activate the liquid.

In one embodiment, the liquid may flow through electrolytic cell 16 as separate streams. For example, the liquid may separate into a pair of sub-streams prior to entering electrolytic cell 16. Alternatively, the liquid may be separated after entering electrolytic cell 16. Furthermore, the liquid may alternatively flow through electrolytic cell 16 as a single stream. As the liquid flows through electrolytic cell 16, the electric field applied across the liquid in electrolysis cell 16 electrochemically activates the liquid, which separates the liquid by collecting positive ions (i.e., cations, H⁺) on one side of an electric circuit and collecting negative ions (i.e., anions, OH⁻) on the opposing side. The liquid having the cations is thereby rendered acidic (i.e., a catholyte EA liquid) and the liquid having the anions is correspondingly rendered alkaline (i.e., an anolyte EA liquid).

The anolyte EA liquid and/or the catholyte EA liquid may then be directed to atomizer 18. In one example, one of the anolyte EA liquid or catholyte EA liquid is directed to atomizer 18, and the other is directed to recovery tank 20, as shown by the broken line in FIG. 1. In another example, both the anolyte and catholyte EA liquids are directed to the steam generator as separate streams or as a single, blended stream.

Atomizer 18 may include any suitable type of atomizing design that renders the EA liquid at least partially aerosol in a gas (e.g., air). Examples of suitable atomizer designs for atomizer 18 include mist sprayers, nebulizers, aerosol generators, piezo atomizers, fogging generators, and combinations thereof. As used herein, the term “aerosol” refers to a suspension of fine liquid droplets in a gas. As such, when rendered aerosol via atomizer 18, the EA liquid may be suspended as fine liquid droplets in the air of the local environment (e.g., the air in a room).

The fine liquid droplets of the aerosol EA liquid may exhibit a variety of different droplet sizes, and may vary depending a variety of factors, such as the surface tension of the EA liquid, the density of the EA liquid, the liquid-to-air ratio in atomizer 18, and the atomizer design used. Examples of suitable average diameters for the fine liquid droplets of the aerosol EA liquid include diameters of about 100 micrometers or less, with particularly suitable average diameters including diameters of about 50 micrometers or less, and with even more particularly suitable average diameters including diameters of about 25 micrometers or less.

The EA liquid, when rendered at least partially aerosol via atomizer 18, is emitted from atomizer 18 (represented by arrows 22) into an environment in which atomizer assembly 10 is retained. For example, the environment may be an indoor location, such as room of a residential, commercial, or industrial building. The aerosol EA liquid is suitable eliminating or reducing the concentration of contaminants in the air and on the surfaces of the environment. Accordingly, atomizer assembly 10 is particularly suitable for use in enclosed or partially enclosed environments to maintain sanitary conditions in such environments. In such environments, the aerosol EA liquid is effective for eliminating or reducing a variety of contaminants, such as microbes, bacteria, fungi, allergens, dust mites, pollen, airborne bioaerosol contaminants, odors, and combinations thereof. Thus, atomizer assembly 10 increases air quality in the environment.

In one embodiment, the aerosol EA liquid eliminates or reduces the concentration of contaminants in the air of the environment. Additionally and/or alternatively, the aerosol EA liquid eliminates or reduces the concentration of contaminants in on one or more surfaces in the environment. This latter embodiment is beneficial for decontaminating the one or more surfaces without contacting the surface(s) with harsh sanitizing solutions.

As discussed above, in one embodiment, the acidic catholyte EA liquid may be directed to atomizer 18, and is rendered at least partially aerosol. As such, the EA liquid rendered aerosol lacks electrons (e.g., oxidizing water) and has a high oxidation reduction potential. This provides antibacterial, an antimicrobial, and/or an antifungal properties to the aerosol EA liquid, which further assists in eliminating or reducing the concentration of contaminants in the air and on the surfaces of the environment.

In additional embodiments, the liquid may also include one or more ingredients, such as odorants, colorants, medicinal ingredients, and combinations thereof. For example, the liquid may include medicinal ingredients for administering the medicinal ingredients to patients in the aerosol EA liquid. Thus, in this embodiment, atomizer assembly 10 may function as a medical nebulizer. Furthermore, atomizer assembly 10 may function as a humidifier to increase the humidity in the environment.

In an alternative example, features of the electrolysis cell 16 and the atomizer 18, such as reservoirs and electrolysis electrodes, can be combined in a single device, such that the working liquid becomes electrolyzed and is atomized within and/or along a combined reservoir, container or flow path. In additional alternative examples, one or both of electrolysis cell 16 and atomizer 18 may include additional reservoirs for retaining liquids for batch operations, or one or both of electrolysis cell 16 and atomizer 18 may be directly fed in a continuous manner.

The arrangement shown in FIG. 1 is provided merely as a non-limiting example. Atomizer assembly 10 can have any other structural and/or functional arrangement. For example, with a self-contained apparatus, reservoir 12 includes a portable vessel that is carried by atomizer assembly 10. In other examples, the reservoir 12 can be external to atomizer assembly 10 and connected through a supply tube. Furthermore, electrolysis cell 16 can be external to atomizer assembly 10. In one example, electrolysis cell 16 is implemented as a stand-alone electrolysis cell, which produces an anolyte EA liquid, a catholyte EA liquid, and/or a combined anolyte and catholyte EA liquid. This EA liquid may then be introduced into atomizer 18 by any suitable method.

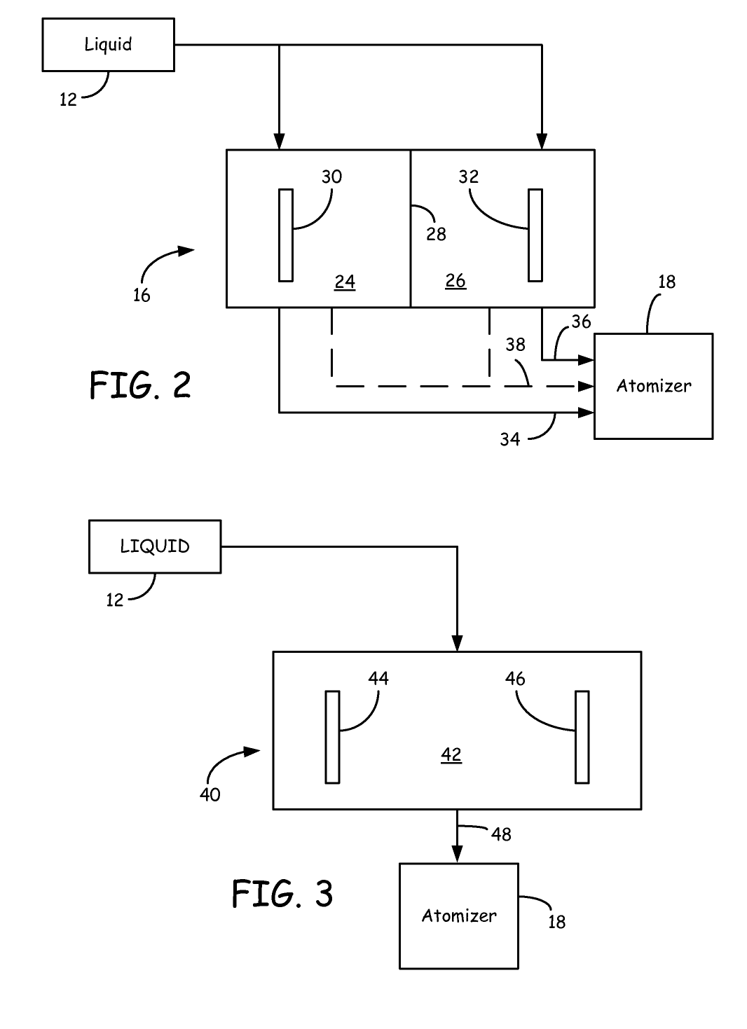

FIG. 2 is a schematic diagram illustrating an electrolysis cell 16 in use with reservoir 12 and atomizer 18 of atomizer assembly 10 (shown in FIG. 1). As discussed above, electrolysis cell 16 receives liquid to be treated from reservoir 12 (and pump 14, shown in FIG. 1). Electrolysis cell 16 includes one or more anode chambers 24 and one or more cathode chambers 26 (known as reaction chambers), which are separated by an ion exchange membrane 28, such as a cation or anion exchange membrane. One or more anode electrodes 30 and cathode electrodes 32 (one of each electrode shown) are disposed in each anode chamber 24 and each cathode chamber 26, respectively. The anode and cathode electrodes 30, 32 can be made from any suitable material, such as titanium and/or titanium coated with a precious metal, such as platinum, or any other suitable electrode material. The electrodes and respective chambers can have any suitable shape and construction. For example, the electrodes can be flat plates, coaxial plates, rods, or a combination thereof. Each electrode can have, for example, a solid construction or can have one or more apertures. In one example, each electrode is formed as a mesh. In addition, multiple electrolysis cells 16 can be coupled in series or in parallel with one another, for example.

The electrodes 30, 32 are electrically connected to opposite terminals of a conventional power supply (not shown). Ion exchange membrane 28 is located between electrodes 30 and 32. The power supply can provide a constant DC output voltage, a pulsed or otherwise modulated DC output voltage, and/or a pulsed or otherwise modulated AC output voltage to the anode and cathode electrodes. The power supply can have any suitable output voltage level, current level, duty cycle or waveform.

For example in one embodiment, the power supply applies the voltage supplied to the plates at a relative steady state. The power supply includes a DC/DC converter that uses a pulse-width modulation (PWM) control scheme to control voltage and current output. Other types of power supplies can also be used, which can be pulsed or not pulsed and at other voltage and power ranges. The parameters are application-specific. The power supply can be embodied within or external to atomizer assembly 10.

During operation, feed water (or other liquid to be treated) is supplied from reservoir 12 to both anode chamber 24 and cathode chamber 26. In the case of a cation exchange membrane, upon application of a DC voltage potential across anode 30 and cathode 32, such as a voltage in a range of about 5 Volts (V) to about 25V, cations originally present in the anode chamber 24 move across the ion-exchange membrane 28 towards cathode 32 while anions in anode chamber 24 move towards anode 30. However, anions present in cathode chamber 26 are not able to pass through the cation-exchange membrane, and therefore remain confined within cathode chamber 26.

While the electrolysis continues, the anions in the liquid bind to the metal atoms (e.g., platinum atoms) at anode 30, and the cations in the liquid bind to the metal atoms (e.g., platinum atoms) at cathode 32. These bound atoms diffuse around in two dimensions on the surfaces of the respective electrodes until they take part in further reactions. Other atoms and polyatomic groups may also bind similarly to the surfaces of anode 30 and cathode 32, and may also subsequently undergo reactions. Molecules such as oxygen (O₂) and hydrogen (H₂) produced at the surfaces may enter small cavities in the liquid phase of the liquid (i.e., bubbles) as gases and/or may become solvated by the liquid phase of the liquid.

Surface tension at a gas-liquid interface is produced by the attraction between the molecules being directed away from the surfaces of anode 30 and cathode 32 as the surface molecules are more attracted to the molecules within the liquid than they are to molecules of the gas at the electrode surfaces. In contrast, molecules of the bulk of the liquid are equally attracted in all directions. Thus, in order to increase the possible interaction energy, surface tension causes the molecules at the electrode surfaces to enter the bulk of the liquid. As a result of the electrolysis process, electrolysis cell 16 electrochemically activates the feed liquid by at least partially utilizing electrolysis and produces the EA liquid in the form of an acidic anolyte composition stream 34 and a basic catholyte composition stream 36.

If desired, the anolyte and catholyte can be generated in different ratios to one another through modifications to the structure of electrolysis cell 16. For example, electrolysis cell 16 can be configured to produce a greater volume of catholyte than anolyte if the primary function of the EA water is cleaning. Alternatively, for example, the cell can be configured to produce a greater volume of anolyte than catholyte if the primary function of the EA water is sanitizing. Also, the concentrations of reactive species in each can be varied.

For example, electrolysis cell 16 can have a 3:2 ratio of cathode plates to anode plates for producing a greater volume of catholyte than anolyte. Each cathode plate is separated from a respective anode plate by a respective ion exchange membrane. Thus, there are three cathode chambers for two anode chambers. This configuration produces roughly 60% catholyte to 40% anolyte. Other ratios can also be used. The polarities can be reversed to achieve roughly 60% anolyte to 40% catholyte.

In addition, water molecules in contact with anode 30 are electrochemically oxidized to oxygen (O₂) and hydrogen ions (H⁺) in the anode chamber 24 while water molecules in contact with the cathode 32 are electrochemically reduced to hydrogen gas (H₂) and hydroxyl ions (OH⁻) in the cathode chamber 26. The hydrogen ions in the anode chamber 24 are allowed to pass through the cation-exchange membrane 28 into the cathode chamber 26 where the hydrogen ions are reduced to hydrogen gas while the oxygen gas in the anode chamber 24 oxygenates the feed water to form the anolyte 34. Furthermore, since regular tap water typically includes sodium chloride and/or other chlorides, the anode 30 oxidizes the chlorides present to form chlorine gas. As a result, a substantial amount of chlorine is produced and the pH of the anolyte composition 34 becomes increasingly acidic over time.

As noted, water molecules in contact with the cathode 32 are electrochemically reduced to hydrogen gas and hydroxyl ions (OH⁻) while cations in the anode chamber 24 pass through the cation-exchange membrane 28 into the cathode chamber 26 when the voltage potential is applied. These cations are available to ionically associate with the hydroxyl ions produced at the cathode 32, while hydrogen gas bubbles form in the liquid. A substantial amount of hydroxyl ions accumulates over time in the cathode chamber 26 and reacts with cations to form basic hydroxides. In addition, the hydroxides remain confined to the cathode chamber 26 since the cation-exchange membrane does not allow the negatively charged hydroxyl ions pass through the cation-exchange membrane. Consequently, a substantial amount of hydroxides is produced in the cathode chamber 26, and the pH of the catholyte composition 36 becomes increasingly alkaline over time.

The electrolysis process in electrolysis cell 16 allows concentrations of reactive species and the formation of metastable ions and radicals in the anode chamber 24 and cathode chamber 26. The electrochemical activation process typically occurs by either electron withdrawal (at anode 30) or electron introduction (at cathode 32), which leads to alteration of physiochemical (including structural, energetic and catalytic) properties of the feed water. It is believed that the feed water (anolyte or catholyte) gets activated in the immediate proximity of the electrode surface where the electric field intensity can reach a very high level. This area can be referred to as an electric double layer (EDL).

As mentioned above, the ion exchange membrane 28 can include a cation exchange membrane (i.e., a proton exchange membrane) or an anion exchange membrane. Suitable cation exchange membranes for membrane 28 include partially and fully fluorinated ionomers, polyaromatic ionomers, and combinations thereof. Examples of suitable commercially available ionomers for membrane 28 include sulfonated tetrafluorethylene copolymers available under the trademark “NAFION” from E.I. du Pont de Nemours and Company, Wilmington, Del.; perfluorinated carboxylic acid ionomers available under the trademark “FLEMION” from Asahi Glass Co., Ltd., Japan; perfluorinated sulfonic acid ionomers available under the trademark “ACIPLEX” Aciplex from Asahi Chemical Industries Co. Ltd., Japan; and combinations thereof. However, any ion exchange membrane can be used in other examples.

In one example, the anolyte and catholyte outputs are blended into a common output stream 38, which is supplied to atomizer 18. As described in Field et al. U.S. Patent Publication No. 2007/0186368, it has been found that the anolyte and catholyte can be blended together within the distribution system of a cleaning apparatus and/or on the surface or item being cleaned while at least temporarily retaining beneficial cleaning and/or sanitizing properties. Although the anolyte and catholyte are blended, they are initially not in equilibrium and therefore temporarily retain their enhanced cleaning and sanitizing properties. It is also believed that the anolyte and catholyte EA liquids retain enhanced cleaning and/or sanitizing properties after being rendered at least partially aerosol by atomizer 18. Thus, the produced aerosol EA liquid has an increased cleaning/sanitizing efficiency.

FIG. 3 illustrates an electrolysis cell 40, which is an alternative to electrolysis cell 16 (shown in FIGS. 1 and 2), and which does not include an ion-selective membrane. Electrolysis cell 40 includes reaction chamber 42, anode 44 and cathode 46. Chamber 42 can be defined by the walls of electrolysis cell 40, by the walls of a container or conduit in which electrodes 44 and 46 are placed, or by the electrodes themselves, for example. Anode 44 and cathode 46 may be made from any suitable material or a combination of materials, such as titanium and/or titanium coated with a precious metal, such as platinum. Anode 44 and cathode 46 are connected to a conventional electrical power supply. In one embodiment, electrolytic cell 40 includes its own container that defines chamber 42 and is located in the flow path of the liquid to be treated.

During operation, liquid is supplied from reservoir 14 (and pump 14, shown in FIG. 1), and is introduced into reaction chamber 42 of electrolysis cell 40. In the embodiment shown in FIG. 3, electrolysis cell 40 does not include an ion exchange membrane that separates reaction products at anode 44 from reaction products at cathode 46. In the example in which tap water is used as the liquid to be treated for use in cleaning, after introducing the water into chamber 42 and applying a voltage potential between anode 44 and cathode 46, water molecules in contact with or near anode 44 are electrochemically oxidized to oxygen (O₂) and hydrogen ions (H⁺) while water molecules in contact or near cathode 46 are electrochemically reduced to hydrogen gas (H₂) and hydroxyl ions (OH⁻). Other reactions can also occur and the particular reactions depend on the components of the liquid. The reaction products from both electrodes are able to mix and form an oxygenated fluid stream 48 (for example) since there is no physical barrier, for example, separating the reaction products from each other. Alternatively, for example, anode 44 can be separated from cathode 44 by using a dielectric barrier such as a non-permeable membrane (not shown) disposed between the anode and cathode.

Referring back to FIG. 1, atomizer assembly 10 can include any suitable control circuit, which can be implemented in hardware, software, or a combination of both, for example. The control circuit can be configured to power and control the operation of pump 14, electrolysis cell 16, and/or atomizer 18. In one example, the control circuit includes a power supply having an output that is coupled to pump 14, electrolysis cell 16 and atomizer 18 and which controls the power delivered to the devices.

In one example the control circuit activates pump 14, electrolysis cell 16 and atomizer 18 in response to actuation of a user switch so that atomizer assembly 10 produces an at least partially aerosol EA liquid in an “on demand” fashion. When the switch is not actuated, pump 14 is in an “off” state and electrolysis cell 16 and atomizer 18 are de-energized. When the switch is actuated to a closed state, the control circuit switches pump 14 to an “on” state and energizes electrolysis cell 16 and atomizer 18. In the “on” state, pump 14 pumps water from reservoir 12 through electrolysis cell 16 and into atomizer 18.

Other activation sequences can also be used. For example, the control circuit can be configured to energize electrolysis cell 16 for a period of time before energizing atomizer 18 in order to allow the reservoir in atomizer 18 to begin to fill with electrochemically activated water before energizing the heating element.

The control circuit can also include an H-bridge, for example, that is capable of selectively reversing the polarity of the voltage applied to electrolysis cell 16 as a function of a control signal generated by the control circuit. For example, the control circuit can be configured to alternate polarity in a predetermined pattern, such as every 5 seconds with a 50% duty cycle. Other frequencies and duty cycles can be used in alternative embodiments. Frequent reversals of polarity can provide a self-cleaning function to the electrodes, which can reduce scaling or build-up of deposits on the electrode surfaces and can extend the life of the electrodes.

The electrodes of the electrolysis cell can be driven with a variety of different voltage and current patterns, depending on the particular application of the cell. It is desirable to limit scaling on the electrodes by periodically reversing the voltage polarity that is applied to the electrodes. Therefore, the terms “anode” and “cathode” and the terms “anolyte” and “catholyte” as used in the description and claims are respectively interchangeable. This tends to repel oppositely-charged scaling deposits.

In one example, the electrodes are driven at one polarity for a specified period of time (e.g., about 5 seconds) and then driven at the reverse polarity for approximately the same period of time. If the anolyte and cathotlyte EA liquids are blended at the outlet of the cell, this process produces essentially one part anolyte EA liquid to one part catholyte EA liquid.

If the outputs are not blended, valving can be used, if desired, to maintain a substantially constant anolyte EA liquid at one outlet and a substantially constant catholyte EA liquid at another outlet, wherein the valving switches states with each switch in polarity so that the anolyte and catholyte liquids are always routed to the same outlet even though the electrolysis chambers switch from anode-to-cathode and vice versa.

If the number of anode electrodes is different than the number of cathode electrodes, e.g., a ratio of 3:2, then the electrolysis cell can be used to produce a greater amount of either anolyte or catholyte, if desired, to emphasize cleaning or sanitizing properties of the produced liquid. For example, if cleaning is to be emphasized, then a greater number of electrodes can be driven to a relatively negative polarity (to produce more catholyte) and a lesser number of electrodes can be driven to the relatively positive polarity (to produce less anolyte). If sanitizing is to be emphasized, then a greater number of electrodes can be driven to the relatively positive polarity (to produce more anolyte) and a lesser number of electrodes can be driven to the relatively negative polarity (to produce less catholyte).

If the anolyte and catholyte outputs are blended into a single output stream prior to dispensing, then the combined anolyte and catholyte output liquid can be tailored to emphasize cleaning over sanitizing or to emphasize sanitizing over cleaning. Although the present disclosure has been described with reference to one or more embodiments, workers skilled in the art will recognize that changes may be made in form and detail without departing from the spirit and scope of the disclosure and/or the appended claims.

1. An atomizer assembly comprising:
an electrolysis cell configured to electrochemically activate a liquid; and
an atomizer configured to render the electrochemically-activated liquid at least partially aerosol.

2. The atomizer assembly of claim 1, wherein the electrolysis cell comprises:
a chamber;
an anode electrode disposed within the chamber, and configured to be electrically connected to a power source; and
a cathode electrode disposed within the chamber, and configured to be electrically connected to the power source.

3. The atomizer assembly of claim 2, wherein the electrolysis cell further comprises an ion exchange membrane disposed between the anode electrode and the cathode electrode.

4. The atomizer assembly of claim 1, wherein the electrochemically-activated liquid comprises acidic water.

5. The atomizer assembly of claim 1, wherein the atomizer is selected from the group consisting of mist sprayers, nebulizers, aerosol generators, piezo atomizers, fogging generators, and combinations thereof.

6. The atomizer assembly of claim 1, wherein the liquid includes one or more ingredients selected from the group consisting of odorants, colorants, medicinal ingredients, and combinations thereof.

7. The atomizer assembly of claim 1, and further comprising one or more components selected from the group consisting of a reservoir configured to retain a supply of the liquid, a liquid pump configured to pump the liquid, a recovery tank, and combinations thereof.

8. A method for decontaminating an environment, the method comprising:
electrochemically activating a liquid; and
rendering the electrochemically-activated liquid at least partially aerosol.

9. The method of claim 8, wherein the electrochemically-activated liquid is rendered at least partially aerosol with at least one atomizer.

10. The method of claim 9, wherein the at least one atomizer is selected from the group consisting of mist sprayers, nebulizers, aerosol generators, piezo atomizers, fogging generators, and combinations thereof.

11. The method of claim 8, wherein electrochemically activating the liquid comprises rendering the liquid acidic.

12. The method of claim 8, wherein the liquid is electrochemically activated in at least one electrolysis cell.

13. The method of claim 8, wherein electrochemically activating the liquid comprises:
introducing a feed liquid into an electrolysis cell, the electrolysis cell having at least one cathode electrode and at least one anode electrode; and
applying a voltage potential across the at least one cathode electrode and the at least one anode electrode to generate the electrochemically-activated liquid from the feed liquid.

14. The method of claim 13, and further comprising maintaining separation of at least two portions of the feed liquid with at least one ion exchange membrane disposed between the at least one cathode electrode and the at least one anode electrode.

15. A method for decontaminating an environment, the method comprising:
introducing a first portion of a feed liquid into an anode chamber of an electrolysis cell;
introducing a second portion of the feed liquid into a cathode chamber of the electrolysis cell;
applying a voltage potential across the first and second portions of the feed water to electrochemically activate the first and second portions of the feed liquid;
feeding at least one of the first and second electrochemically-activated portions of the feed liquid to an atomizer; and
rendering the at least one electrochemically-activated portion at least partially aerosol.

16. The method of claim 15, and further comprising maintaining separation of the anode chamber and the cathode chamber within the electrolysis cell with an ion exchange membrane.

17. The method of claim 15, the at least one electrochemically-activated portion comprises acidic water.

18. The method of claim 15, wherein the at least one electrochemically-activated portion is rendered at least partially aerosol with at least one atomizer.

19. The method of claim 18, wherein the at least one atomizer is selected from the group consisting of mist sprayers, nebulizers, aerosol generators, piezo atomizers, fogging generators, and combinations thereof.

20. The method of claim 15, wherein the at least one electrochemically-activated portion that is rendered at least partially aerosol is configured to eliminate or reduce contaminants selected from the group microbes, bacteria, fungi, allergens, dust mites, pollen, airborne bioaerosol contaminants, odors, and combinations thereof.
